(19) United States
(12) Patent Application Publication
(10) Pub. No.: US 2007/0039211 A1
(43) Pub. Date: Feb. 22, 2007

(54) Orthotic insert having recess for therapeutic patch

(76) Inventor: Thomas Pichler, San Diego, CA (US)

Correspondence Address: PETER K HAHN;LUCE, FORWARD, HAMILTON, SCRIPPS, LLP., 600 WEST BROADWAY, SUITE 2600, SAN DIEGO, CA, 92101, US

(21) Appl. No.: 11/205,624

(22) Filed: Aug. 17, 2005

(51) Int. Cl. A61F 5/14

(52) U.S. Cl. 36/140

(57) ABSTRACT

The present invention provides an orthotic insert for a shoe, the orthotic insert comprising a heel region, an arch region and a recess for receiving a therapeutic patch for the treatment of a shoe wearer's foot, wherein the therapeutic patch provides foot treatment in the form of continuous electromagnetic energy for treating and restoring damaged foot tissue cells.

### FIELD OF THE INVENTION

The present invention is directed to an orthotic insert having a recess for a therapeutic patch, and more particularly to an orthotic shoe insert having a recess for a therapeutic patch for delivering pulsed electromagnetic field (PEMF) therapy to promote the healing of foot tissue.

### BACKGROUND OF THE INVENTION

Millions of humans suffer from various foot and related back problems stemming from the impact of walking and running while wearing shoes. Custom orthotic inserts have been developed to provide some protection to the shoe wearer's feet in the form of cushioning and shock absorption. Conventional orthotic inserts seek to provide a custom fit of the shoes around the contours of the shoe wearer's feet to provide support and stability.

Typical orthotic inserts are orthopedic devices designed to protect, support, align, correct, or improve the function of the foot. Some orthotic inserts are designed to hinder excessive lateral movement such as pronation or rolling of the foot. Other inserts are configured to compensate and correct such abnormal movement. Additionally, it is well recognized by podiatrists and chiropractors that many health problems including spinal and joint problems can be improved or corrected by properly constructed orthotic inserts.

One drawback of conventional orthotic inserts is that they are not designed to receive a patch that eliminates the need for a trained operator and recurring office visits for treatment. A further drawback of conventional orthotic inserts is that they do not provide a cutout area for receiving a patch for providing electromagnetic therapy for the wearer's feet.

In view of the above, there exists a need for an orthotic insert including a recess for receiving a therapeutic patch for treatment of the wearer's foot.

There further exists a need for an orthotic insert that improves patient compliance by providing a quicker and more convenient application of foot therapy.

Additionally, there exists a need for an orthotic insert that reduces the cost of foot therapy to a small fraction of conventional therapy costs.

There also exists a need for an orthotic insert that eliminates the need for a trained operator and recurring office visits for foot treatment

There further exists a need for an orthotic insert designed to receive a patch that eliminates the need for a trained operator and recurring office visits for treatment.

Additionally, there exists a need for an orthotic insert having a cutout area for receiving a patch for providing electromagnetic foot therapy.

There also exists a need for an orthotic insert including a recess for receiving a miniaturized, portable and inexpensive patch for treatment of the wearer's foot.

There further exists a need for an orthotic insert including an orthotic patch that utilizes the body's own electrical properties and reduces power requirements to a 3-volt DC battery.

### SUMMARY OF THE INVENTION

In view of the foregoing, it is an object of the present invention to provide an orthotic insert including a recess for receiving a therapeutic patch for treatment of the wearer's foot.

It is another object of the present invention to provide an orthotic insert that improves patient compliance by providing a quicker and more convenient application of foot therapy.

It is a further object of the present invention to provide an orthotic insert that reduces the cost of foot therapy to a small fraction of conventional therapy costs.

Additionally, it is an object of the present invention to provide an orthotic insert that eliminates the need for a trained operator and recurring office visits for foot treatment

It is another object of the present invention to provide an orthotic insert designed to receive a patch that eliminates the need for a trained operator and recurring office visits for treatment.

It is a further object of the present invention to provide an orthotic insert having a cutout area for receiving a patch for providing electromagnetic foot therapy.

It is yet another object of the present invention to provide an orthotic insert including a recess for receiving a miniaturized, portable and inexpensive patch for treatment of the wearer's foot.

Additionally, it is an object of the present invention to provide an orthotic insert including an orthotic patch that utilizes the body's own electrical properties and reduces power requirements to a 3-volt DC battery.

One aspect of the present invention involves an orthotic insert for a shoe, the insert comprising a heel region, an arch region and a recess for receiving a therapeutic patch for treatment of a shoe wearer's foot, wherein the orthotic insert is custom sized and dimensioned to conform to the shape and size of the shoe and the shoe wearer's foot. The shape and size of the recess is determined by the shape and size of the corresponding therapeutic patch. The recess may be disposed in any region of the orthotic insert including the heel region, the arch region or the metatarsal region.

An additional aspect of the invention involves an orthotic insert for a shoe, the insert comprising a heel region, an arch region and a recess for receiving a therapeutic patch for treatment of a shoe wearer's foot and an antenna for channeling the therapeutic energy to a region of the orthotic insert that is spatially offset from the recess. For example, the antenna may be used to channel the therapeutic energy from the arch region to the heel region of the orthotic insert for treatment of the shoe wearer's heel. According to a preferred embodiment of the invention, the antenna is tuned for electromagnetic energy in the frequency range of 27+/0.5 Mhz.

A further aspect of the present invention involves an orthotic insert for a shoe, the insert comprising a heel region, an arch region and a recess for receiving a therapeutic patch, wherein the therapeutic patch comprises a PEMF therapy device that provides foot treatment in the form of continuous electromagnetic energy for treating and restoring damaged cells. The therapeutic patch may be used to treat a foot ailment chosen from the group consisting of: non-union bone fractures that have not satisfactorily healed within a predetermined time from the date of injury; delayed union bone fractures that have not satisfactorily healed within a predetermined time from the date of injury; soft tissue injuries that are slow in healing; ligament injuries; tendon injuries; heel pain; arch pain; foot pain; and heel spurs.

Yet another aspect of the present invention involves an orthotic insert for a shoe, the insert comprising a heel region, an arch region and a recess for receiving a therapeutic patch, wherein the therapeutic patch is securely affixed within the recess using an adhesive. In addition, the therapeutic patch may be encapsulated to reduce the vulnerability of the device to fluids, moisture, static charges and particulate matter. According to a preferred embodiment of the invention, the therapeutic patch is powered using a 3-volt DC battery.

According to some embodiments of the invention, the orthotic insert may be provided with a plurality of thermosensors for determining the temperature at various regions of the foot, such as the heel region, the arch region and the metatarsal region. These thermosensors may be built into the top layer of the orthotic in order to locate hotspots and regions of lower temperatures, which are often the first indicators of ulcer formation in diabetic patients

### BRIEF DESCRIPTION OF THE DRAWINGS

FIG. 1 is a top view of an orthotic shoe insert having a recess for receiving a therapeutic device for treatment of a shoe wearer's foot, in accordance with the principles of the present invention;

FIG. 2 is a cross sectional view of the orthotic insert of FIG. 1 disposed within a conventional shoe;

FIG. 3 is a top view of an alternative embodiment of the orthotic shoe insert of FIG. 1;

FIG. 4 is a top view of an alternative embodiment of the orthotic shoe insert of FIG. 1;

FIG. 5 is a top view of an alternative embodiment of the orthotic shoe insert of FIG. 1; and

FIGS. 6 and 7 are top and exploded perspective views, respectfully, of an exemplary therapeutic patch in accordance with the principles of the present invention.

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

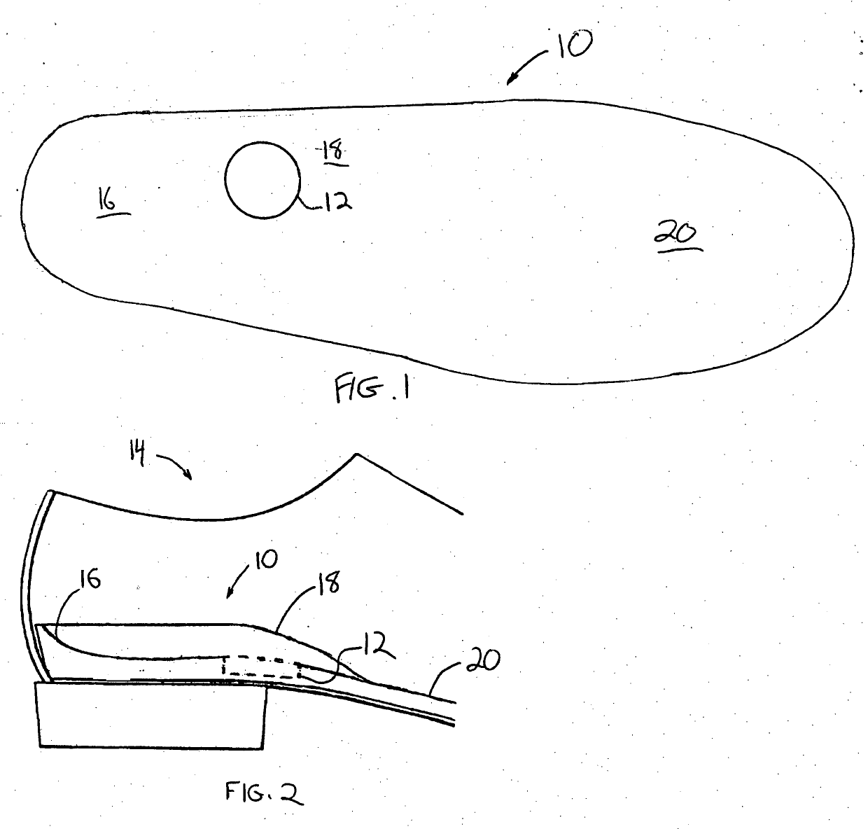

Referring to FIGS. 1 and 2, the present invention is directed to an orthotic shoe insert 10 having a cutout or recess 12 for receiving a therapeutic device for treatment of a shoe wearer's foot. In particular, the therapeutic device may provide a therapeutic microcurrent such as an electromagnetic field for treating a region of the shoe wearer's foot such as the heel or the arch. Advantageously, the present invention provides an orthotic insert that reduces the cost of foot therapy to a small fraction of conventional foot therapy costs by eliminating the need for recurring office visits for foot treatment. In addition, patient compliance is improved by providing a quicker and more convenient application of foot therapy. According to some embodiments of the invention, the therapeutic patch utilizes the shoe wearer's own electrical properties to reduce power requirements to a 3-volt DC battery.

Referring to FIG. 2, orthotic shoe insert 10 is configured to be inserted within conventional shoe 14 and worn by the shoe wearer during normal use of the shoe. In the illustrated embodiment, exemplary orthotic shoe insert 10 comprises a substantially cup-shaped heel region 16, an arch region 18 of increased and variable thickness and a metatarsal region 20 that is substantially planar. According to other embodiments, the metatarsal region is not provided. The orthotic insert may be custom sized and dimensioned to conform to the shape and size of a particular foot and/or shoe. The custom orthotic insert may be configured to prevent protect, support, align, correct, or improve the function of the foot. By way of example, orthotic insert 10 may be configured to hinder or correct excessive lateral movement or rolling of the foot, or to alleviate related problems such as spinal and joint trauma. As would be understood by those of ordinary skill in the art, the orthotic inserts described herein may comprise many custom shapes, sizes and dimensions without departing from the scope of the invention.

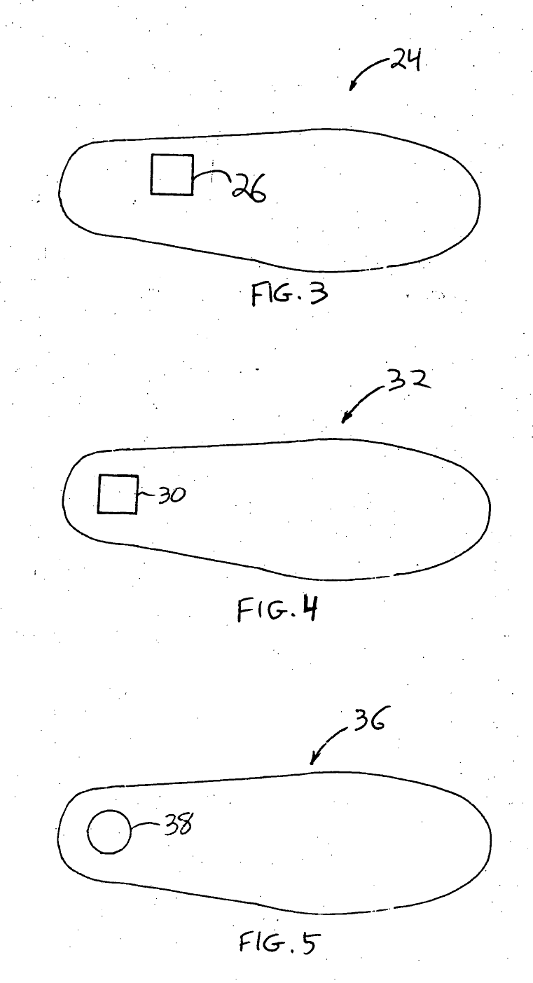

Recess 12 preferably is sized and dimensioned to securely receive a therapeutic patch for treatment of the shoe wearer's foot such that the recess functions as a housing for the therapeutic patch. In the illustrated embodiment, recess is substantially circular in cross section and disposed generally in arch region 18 of orthotic insert 10. As would be appreciated by those of ordinary skill in the art, recess 12 may comprise various other shapes, dimensions and sizes without departing from the scope of the present invention. For example, as depicted in FIG. 3, orthotic shoe insert 24 includes recess 26 that is substantially rectangular in cross section, wherein the recess is generally disposed in the arch region of the insert. According to additional embodiments of the invention, the recess may include any other cross sectional shape such as substantially square, rectilinear, triangular, polygonal, curvilinear and combinations of these shapes.

With further reference to FIG. 1, recess 12 located substantially in the arch region 18 of the orthotic insert. A therapeutic patch that is disposed within the recess may be used to provide treatment for the shoe wearer's arch. Alternatively, the therapeutic patch may include an antenna to channel electromagnetic energy to the heel region for treatment of the shoe wearer's heel. In this manner, the recess 12 located substantially in arch region 18 is spatially offset from the treatment area in heel region 16. According to further embodiments of the invention, the antenna may be configured to channel the electromagnetic energy to any other area of the orthotic insert, including the arch region and the metatarsal region. As depicted in FIG. 2, arch region 18 comprises an area of increased thickness, thereby providing ample space for the recess disposed therein without substantially affecting the stability and support of the insert, particularly in the arch region.

According to further embodiments of the invention, the recess may be disposed in other regions of the orthotic insert such as the heel region or the metatarsal region. By way of example, as depicted in FIG. 4, recess 30 comprises a substantially rectilinear cutout disposed generally in the heel region of orthotic insert 32. A therapeutic patch may be disposed within recess 30 for treatment of the heel area of the shoe wearer's foot. Alternatively, an antenna may be used to channel the therapeutic energy from the patch to other areas of the orthotic insert such as the arch region and the metatarsal region. Referring to FIG. 5, the illustrated orthotic insert 36 includes a recess 38 that is substantially circular in cross section and is disposed generally in the heel region of the insert. Of course, the recess may comprise many additional shapes, dimensions, sizes and locations within the insert without departing from the scope of the present invention.

According to the preferred embodiment of the invention, the therapeutic patch comprises an inexpensive, miniaturized, removable device that provides foot treatment such as in the form of continuous electromagnetic energy for treating and restoring damaged cells. The electromagnetic orthotic patch may be employed as part of a convenient 24-hour treatment, wherein the length of treatment is limited to the time when the patient is wearing the orthotic insert. The effectiveness of electromagnetic therapy may be improved by extending the treatment duration. In general, lower power electromagnetic radiation may be applied for a longer period of time than higher power electromagnetic radiation. The treatment of acute conditions may require a higher repetition with narrower pulses than for the treatment of pain. Advantageously, the therapeutic patch may be removed from the recess and replaced with a new therapeutic patch having a fresh battery.

In accordance with the principles of the invention, the therapeutic patch may comprise a pulsed electromagnetic field (PEMF) therapy device that is employed to treat various regions of the foot. The PEMF therapy device is embedded within the orthotic insert for the treatment of many foot ailments including, but not limited to: (1) non-union bone fractures that have not satisfactorily healed within a predetermined time from the date of injury; (2) delayed union bone fractures that have not satisfactorily healed within a predetermined time from the date of injury; (3) soft tissue injuries that are slow in healing; (4) ligament injuries; (4) tendon injuries; (5) heel pain; (6) arch pain; (7) foot pain; (8) heel spurs; and (9) other foot problems. As would be appreciated by those of ordinary skill in the art, the therapeutic patch may comprise other forms of therapeutic energy without departing from the scope of the present invention.

PEMF therapy systems are commonly used to treat resistant injuries of the musculoskeletal system using low-energy, time-varying magnetic fields. During therapy, an electromagnetic transducer coil is placed proximal to the injury such that pulsing of the transducer coil produces an electromagnetic field that penetrates the damaged body tissue. Traditional PEMF therapy must be administered by trained operator using bulky and expensive PEMF machines. Such therapy is undesirable due to the following factors: (1) the high cost of large PEMF machines; (2) the required frequency of therapy; and (3) the fact that a trained operator must administer treatment.

The present invention provides a therapeutic patch for an orthotic insert for delivering energy (e.g., PEMF therapy) to promote the healing of body tissue. In operation, a low-level current is gently pulsed into the treatment area for treatment of damaged tissue. Advantageously, the low-level energy is non-discernible even though the patch is continuously working to treat the damaged tissue. Moreover, the low-level energy produced by the PEMF device comprises an extremely safe form of therapeutic energy. The components of the therapeutic electromagnetic patch preferably are integrated into a miniaturized control circuit chip embedded within the orthotic insert. Specifically, the patient must merely insert the orthotics into their shoes, rather than taping or otherwise adhering the electromagnetic patch to the damaged tissue area.

According to further embodiments of the present invention, the orthotic insert may be provided with a plurality of thermosensors for determining the temperature at various regions of the foot, such as the heel region, the arch region and the metatarsal region. The thermosensors may be built into the top layer of the orthotic in order to locate hotspots and regions of lower temperatures, which are often the first indicators of ulcer formation in diabetic patients

Foot tissue cells are tiny, electrically charged machines that may become traumatized and require treatment using therapeutic energy. Traumatized tissue cells have diminished charges such that normal cell functions and operations may be interrupted or shut down entirely. Chemical signals that cause inflammation are released in proximity to damaged tissue, frequently resulting in pain and inhibiting the cell communication necessary for healing to begin. The electromagnetic patch of the invention induces a mild electrical current for treating the damaged foot tissue cells. The electrical current may have one or more of the following beneficial effects: (1) recharging the cell; (2) increasing blood flow; (3) inhibiting the release of pain and inflammatory mediators; and (4) re-establishing normal cell interaction. As a result, the damaged tissue area heals faster and experiences less swelling, pain and bruising.

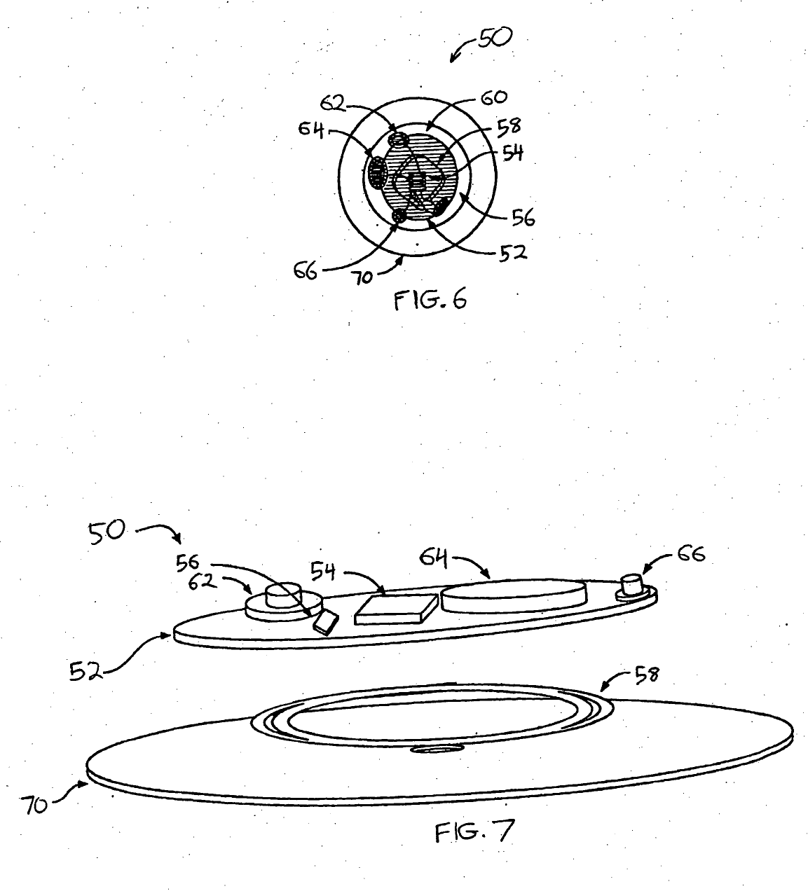

Referring to FIGS. 6 and 7, exemplary therapeutic electromagnetic patch 50 is a PEMF therapy device comprising circuit board 52, control circuit chip 54, tuning coil 56, antenna 58, activator 62, power source 64 and indicator light 66. The therapeutic patch may also include a pad 70 to cushion the shoe wearer's foot during use of the orthotic insert. Therapeutic patch 50 is adapted to be inserted within the recess with the circuit board facing down such that pad 70 is substantially flush with the top surface of the orthotic insert. The patch may be securely affixed within the recess using an adhesive. In addition, the patch may be encapsulated to reduce the vulnerability of the device to fluids, moisture, static charges and particulate matter. Suitable encapsulants include insulating resins such as epoxies, polyurethanes, and polyesters, which may be cast into the recess containing the patch for the purpose of insulating, protecting and stabilizing the patch components.

With continued reference to FIGS. 6 and 7, circuit board 52 is used to mount the various electrical components of the patch and provide electrical coupling therebetween. Control circuit chip 54 provides operational functionality for the patch. Power source 64 may comprise a battery or electrochemical cell used to power the therapeutic electromagnetic device. According to the preferred embodiment of the invention, power source 64 comprises a 3-volt DC battery. Activator 62 may include a switch for activating the device such that activation is achieved by use of pressure, light or electronic signal. Alternatively, therapeutic electromagnetic patch 50 may be automatically activated when power source 64 is applied.

Antenna 58 radiates electromagnetic energy. According some embodiments of the invention, the antenna is employed to channel the therapeutic electromagnetic energy in the proximity of the therapeutic patch. According to other embodiments, antenna 58 may be used to channel the therapeutic electromagnetic energy to a location that is spatially offset from the therapeutic patch. For example, as depicted in FIG. 1, the recess 12 is disposed in arch region 18 of orthotic insert 10, whereas the antenna may be disposed in heel region 16 to channel the therapeutic energy toward the shoe wearer's heel.

In accordance with the preferred embodiment of the invention, the antenna is tuned for electromagnetic energy in the frequency range of 27+/0.5 Mhz. Tuning coil 56 may be used to match the impedance of antenna 58 to the RF signal generator within control circuit chip 54. Indicator light 66 is provided to indicate when the therapeutic electromagnetic device is active. By way of example, indicator light 66 may comprise one or more light emitting diodes (LEDs) coupled to an electromagnetic field detector in control circuit chip 54.

Thus, it is seen that an orthotic insert having a recess for a therapeutic patch is provided. One skilled in the art will appreciate that the present invention can be practiced by other than the various embodiments and preferred embodiments, which are presented in this description for purposes of illustration and not of limitation, and the present invention is limited only by the claims that follow. It is noted that equivalents for the particular embodiments discussed in this description may practice the invention as well.

What is claimed is:

1. An orthotic insert for a shoe, comprising:
a heel region;
an arch region; and
a recess for receiving a therapeutic patch for treatment of a shoe wearer's foot.

2. The orthotic insert of claim 1, wherein the orthotic insert is custom sized and dimensioned to conform to the shape and size of the shoe and the shoe wearer's foot.

3. The orthotic insert of claim 1, wherein the recess is substantially circular in cross section.

4. The orthotic insert of claim 1, wherein the recess is a substantially rectilinear cutout.

5. The orthotic insert of claim 1, wherein the recess is disposed generally in the arch region of the orthotic insert.

6. The orthotic insert of claim 1, wherein the recess is disposed generally in the heel region of the orthotic insert.

7. The orthotic insert of claim 1, wherein the cross sectional shape of the recess is selected from the group consisting of: rectangular; square; rectilinear; triangular; polygonal; curvilinear; and combinations thereof.

8. The orthotic insert of claim 1, wherein the recess is located substantially in the arch region of the orthotic insert for treatment of the shoe wearer's arch.

9. The orthotic insert of claim 5, further comprising an antenna for channeling the therapeutic energy to a region of the orthotic insert that is spatially offset from the recess.

10. The orthotic insert of claim 10, wherein the antenna is used to channel the therapeutic energy to the heel region of the orthotic insert for treatment of the shoe wearer's heel.

11. The orthotic insert of claim 1, wherein the therapeutic patch provides foot treatment in the form of continuous electromagnetic energy for treating and restoring damaged cells.

12. The orthotic insert of claim 1, wherein the therapeutic patch comprises a PEMF therapy device for providing foot treatment.

13. The orthotic insert of claim 1, wherein the therapeutic patch is used to treat a foot ailment chosen from the group consisting of: non-union bone fractures that have not satisfactorily healed within a predetermined time from the date of injury; delayed union bone fractures that have not satisfactorily healed within a predetermined time from the date of injury; soft tissue injuries that are slow in healing; ligament injuries; tendon injuries; heel pain; arch pain; foot pain; and heel spurs.

14. The orthotic insert of claim 1, wherein the therapeutic patch is securely affixed within the recess using an adhesive.

15. The orthotic insert of claim 1, wherein the therapeutic patch is encapsulated to reduce the vulnerability of the device to fluids, moisture, static charges and particulate matter.

16. The orthotic insert of claim 1, wherein the therapeutic patch may be removed from the recess and replaced with a new therapeutic patch having a fresh battery.

17. The orthotic insert of claim 1, wherein the therapeutic patch comprises:
an antenna that is tuned for electromagnetic energy in the frequency range of 27+/0.5 Mhz; and
an indicator light for indicating when the therapeutic patch is active.

18. An orthotic insert for a shoe, comprising:
a heel region;
an arch region; and
a recess for receiving a therapeutic patch for treatment of a shoe wearer's foot;
wherein the therapeutic patch provides foot treatment in the form of continuous electromagnetic energy for treating and restoring damaged foot tissue cells.

19. The orthotic insert of claim 18, further comprising an antenna for channeling the therapeutic energy to a region of the orthotic insert that is spatially offset from the recess.

20. An orthotic insert for a shoe, comprising:
a heel region;
an arch region; and
a recess for receiving a therapeutic patch for treatment of a shoe wearer's foot;
wherein the therapeutic patch comprises a PEMF therapy device for providing foot treatment in the form of continuous electromagnetic energy for treating and restoring damaged foot tissue cells.

21. The orthotic insert of claim 20, further comprising a plurality of thermosensors built into the insert for locating hotspots and regions of lower temperatures.
